(19) United States
(12) Patent Application Publication
(10) Pub. No.: US 2010/0239505 A1
(43) Pub. Date: Sep. 23, 2010

(54) APPARATUS WITH AN ECHOGENIC COATING AND ECHOGENIC LAYER

(75) Inventors: Rudolf REICHL, Metzingen (DE); Bernhard SCHRÖDER, Lichtenstein (DE)

Correspondence Address: DENNISON, SCHULTZ & MACDONALD, 1727 KING STREET, SUITE 105, ALEXANDRIA, VA, 22314, US

(73) Assignee: RUGER MEDICAL GMBH, Felsberg (DE)

(21) Appl. No.: 12/724,489

(22) Filed: Mar. 16, 2010

(30) Foreign Application Priority Data
Mar. 17, 2009 (DE) 20 2009 001 974.8

(51) Int. Cl. A61K 49/22; A61M 25/00; A61M 5/00; A61F 2/82

(52) U.S. Cl. 424/9.52; 604/523; 604/272; 623/1.46

(57) ABSTRACT

An apparatus having an echogenic coating of a polymer with hollow spaces present in the polymer. In order to achieve an echogenicity that supplies largely isotropic ultrasonic echo images, the hollow spaces should be microhollow spheres embedded in the polymer.

### BACKGROUND OF THE INVENTION

The invention relates to an apparatus such as a catheter, cannula or needle with an echogenic coating of a polymer with hollow spaces present in the latter. The invention also relates to an echogenic layer comprising a polymer with hollow spaces present in it.

In order to be able to check a proper setting of, e.g., cannulas, catheters, needles or stents in a body, the providing of appropriate biomedical apparatuses with a subtractive structure or with a layer that has echogenic properties is known.

Subtractive structures can be achieved by processing the apparatus surface. Thus, WO-A-01/87177 discloses a medical apparatus, whose surface has corrugated indents.

According to U.S. Pat. No. 4,869,259 a medical instrument is roughened by blasting it with particles with diameters of 50 μm in order to achieve echogenic properties. However, corresponding subtractive structures have the disadvantage that in the case of rough boundary surfaces that are not arranged vertically to the ultrasonic jet an echo can be registered by back scattering of a diffuse jet cone. The scatter changes in accordance with the diameter of the scatter center. In the geometric area in which the diameter of the scatter center is much larger than the wavelength of the ultrasound the scatter is strong, i.e., in the frequency range between 1 MHz and 10 MHz in structures between 0.15 mm and 1.54 mm.

There is also the possibility of introducing holes into a cannula whose diameter is approximately equal to the wavelength of the ultrasound (U.S. Pat. No. 4,997,897).

The known biomedical apparatuses that are mechanically worked on the surface side in order to form a subtractive structure display a pronounced angular dependency. The reflection of the ultrasound is a function of the position of the apparatus such as cannula relative to the incident ultrasound. At angles used in the practice there is no ability to make a clean demarcation at rather high angles.

Heterogeneous layer systems are applied to a considerable extent onto medical apparatuses and devices that make use of the impedance of the ultrasound in different phases. In the case of the reflection of the sound on smooth boundary surfaces between areas with different impedance the coefficient of reflection, that is, the relationship of reflected to incident sound intensity, is the square of the quotient of difference and thus of the individual impedances. This means that the greater the difference of impedance, the greater the reflection.

Corresponding layer systems can comprise a matrix of a polymeric material such as polyurethane in which compressible enclosed gas bubbles form during heating that result in a reflection of the ultrasound (EP-B-0 941 121).

A medical needle according to U.S. Pat. No. 5,383,466 has a surface layer in which gas bubbles are contained in a matrix of polymer.

A medical instrument according to U.S. Pat. No. 6,306,094 has a coating on the outside in which discrete movable bubbles are produced upon a reaction with a reactant.

A surgical instrument according to U.S. Pat. No. 6,749,554 is provided with a coating comprising a matrix with contrast-reinforcing elements that for their part change their reflection properties as a function of the temperature.

If echogenic properties are achieved by coatings, in particular by embedded foreign particles—in particular gas bubbles with the greatest reflection of the ultrasound—then an uneven reflection occurs conditioned by the production process, because it is not possible to produce gas bubbles with uniform dimensions for the optimal ultrasonic reflection since the gas bubbles are produced by chemical reaction and vary greatly in diameter.

### SUMMARY OF THE INVENTION

It is the object of the present invention to further develop an apparatus as well as a layer of the initially cited type in such a manner that an echogenicity can be achieved that supplies largely isotropic ultrasonic echo images at angles used in practice, in particular in the medical area when using the apparatus on patients. Also, a reproducible coating should be made available. The coating itself should be able to produced economically in standard production processes in particular.

The invention achieves these objects by providing as the hollow spaces, microhollow spheres embedded in the polymer.

According to the invention, an apparatus with a homogeneous echogenic coating is provided that makes isotropic ultrasonic echo images possible. This applies in particular to invasive medical products and to all angles used in practice. Invasive medical products are, e.g., catheters, cannulas, puncture needles.

An optimal ultrasonic reflection takes place independently of the angle of incidence of the ultrasound.

In particular, it is provided that the polymer is a dispersion of, or contains, polyurethane.

A biocompatible commercial polyurethane dispersion lacquer can be used that is provided with an addition of microhollow spheres with a defined diameter in order to then be applied onto the apparatus. In particular, microhollow spheres filled with isobutane are supplied to the polymer.

The microhollow spheres themselves should be formed of vinylidene chloride.

Preferred products can then be achieved if the microhollow spheres have a diameter between 5 μm and 50 μm.

The layer density of the polymer filled with the microhollow spheres, that is, in particular polyurethane dispersion lacquer, should be between 50 μm and 70 μm.

The degree of filling of the polymer such as polyurethane lacquer should be between 5% by vol. and 25% by vol., especially approximately 10% by vol. if the diameter of the hollow spheres is in the range of 20 μm.

In particular, it is provided that the amount of the microhollow spheres, in particular vinylidine chloride microhollow spheres filled with isobutane is 3.5 wt % at a particle size of approximately 20 μm.

The density of the vinylidine chloride microhollow spheres filled with isobutane is approximately 0.7 g/cm³.

A suitable commercial polyurethane dispersion lacquer is, e.g., Bayhydrol PR340 of Bayer MaterialScience, and the specification relative to this product is incorporated by reference.

In order to produce the initial material, vinylidine chloride microhollow spheres filled, e.g., with isobutane are mixed by machine into a commercial polyurethane dispersion in which the weight component of the spheres is in the range of 3 wt % to 4 wt %, especially approximately 3.5 wt %. Before the application of the dispersion onto the apparatus such as the invasive medical product such as catheter, cannula or stent, at first a vapor degreasing in a solvent cleaning bath should take place. Then, an extremely fine cleaning and activation of the surface by plasma treatment is provided. In the case of catheters, the vapor degreasing can be optionally eliminated.

The dispersion can then be applied by spraying in several work passages with intermediate drying. The drying itself should take place at a temperature of approximately 100° C. over a time of approximately 20 minutes-30 minutes, preferably approximately 25 minutes.

An echogenic layer comprising a polymer with hollow spaces embedded in it is distinguished in that the hollow spaces are microhollow spheres embedded in the polymer. The polymer should be a dispersion of polyurethane or contain it.

The microhollow spheres themselves have, in particular, a diameter in the range between 5 μm and 50 μm.

In order to achieve desired isotropic ultrasonic echo images, namely, at angles customarily used in the medical field when setting catheters or puncture needles, the volumetric amount of the hollow spheres should be between 5% by vol. and 25% by vol. The microhollow spheres themselves are preferably filled with a gas such as isobutane.

The microhollow spheres can be formed of vinylidene chloride.

### BRIEF DESCRIPTION OF THE DRAWINGS

Further details, advantages and features of the invention result not only from the claims, the features to be gathered from them by themselves and/or in combination, but also from the following description of a preferred exemplary embodiment shown in the drawings, in which:

FIG. 1 shows a section of a cannula;

FIG. 2 shows a test structure; and

FIG. 3 shows an ultrasonic photograph.

### DESCRIPTION OF THE PREFERRED EMBODIMENTS

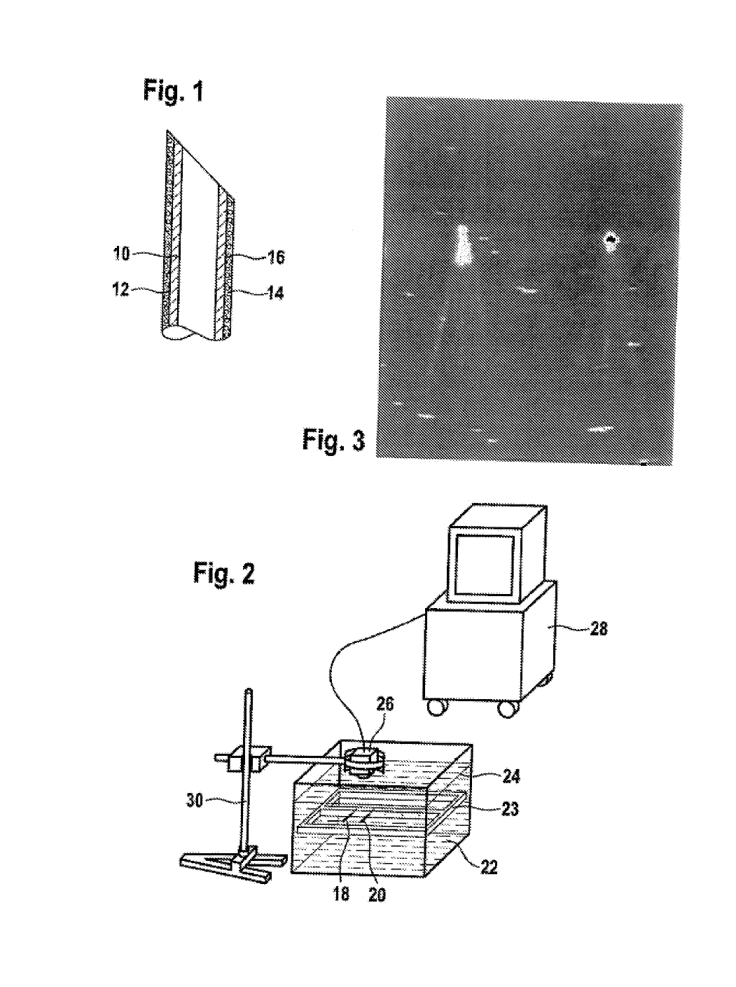

FIG. 1 shows a biomedical apparatus in the form of a cannula 10 in a purely diagrammatic form and in section, having applied on its outer surface 12 an echogenic layer 14 that is comprised in the exemplary embodiment of a polyurethane dispersion lacquer with vinylidine chloride microhollow spheres 16 filled with isobutane, in an amount of 3.5 wt % of the coating. Microhollow spheres 16 have a particle size of approximately 20 μm and a density of approximately 0.7 g/cm³.

Before application of the layer 14, the cannula 10, that is, its surface 12 is first degreased by vapor in a solvent cleaning bath in order to then achieve an extremely fine cleaning and activation of the surface by plasma pretreatment. Then, the modified polyurethane dispersion, that is, a commercial polyurethane dispersion provided with vinylidine chloride microhollow spheres filled with isobutane is applied in several work passages with intermediate drying on surface 12. The individual drying procedures amounted to approximately 20 minutes-30 minutes at a temperature of 100° C.

In order to check the echogenicity of cannula 10 in comparison to a cannula provided with a subtractive structure and roughened on the surface, a cannula 18 with the coating in accordance with the invention and a reference cannula 20 were suspended on gimbals (gimbal suspension 23), as shown in FIG. 2. Care was taken that the ground section of the needles 18, 20 faced the bottom of a basin filled with water. The distance between the two needles 18, 20 was approximately 2 cm. Basin 22 was filled with water until the distance of needles 18, 20 to the water surface 24 was approximately 2 cm.

An ultrasonic head 26 was arranged above water surface 24 and above needles 18, 20. The ultrasonic head was the small sound head of the ultrasonic diagnosing device ACUSON Antares Premium Edition that is customarily used in the examination of surface muscles. In order to obtain the most comparable and reproducible results possible, head 26 was fastened on a stand 30. On the ultrasonic diagnosing device 28, the muscle protocol was selected with a frequency of 11.43 MHz. Needles 18, 20 were examined at the angles 0°, 10°, 20° and 30° to the horizontal plane and sound head 26 was arranged transversely to needles 18, 20. The focus was adjusted in such a manner that it was approximately in the needle plane.

The angle runs between the normal originating from the longitudinal axis of the cannula and the direction of the sound. In practice, the sound direction should run along the normal. Angles up to 45° are possible. However, the angle should preferably not exceed 30° in order to be able to make use of the echogenic properties in accordance with the invention to a sufficient extent.

The comparison tests showed that the visibility of the needle coated in accordance with the invention, that is, the needle 18 in the ultrasonic field (echogenicity) is better at the angles used in practice than that of the reference needle 20. The echogenicity is consequently much better. Also, fewer artifacts occur.

FIG. 3 is an ultrasonic photograph obtained with the test arrangement shown in FIG. 2. The photograph is a false color representation of an ultrasonic picture in which needles 18, 20 were inclined by 10° to ultrasonic head 26. On the right, the needle 18 coated in accordance with the invention is shown in comparison to the reference needle 20 (left). The needle 18 coated in accordance with the invention is clearly recognizable.

What is claimed is:

1. An apparatus (10) such as a catheter, cannula, needle or stent, with an echogenic coating (4) of a polymer with hollow spaces (16) present in the polymer, wherein the hollow spaces (16) are microhollow spheres embedded in the polymer.

2. The apparatus according to claim 1, wherein the polymer is a dispersion of polyurethane or contains polyurethane.

3. The apparatus according to claim 1, wherein the microhollow spheres (16) have a diameter D with 5 μm≦D≦50 μm.

4. The apparatus according to claim 3, wherein the microhollow spheres (16) have a diameter D with D≈20 μm.

5. The apparatus according to claim 1, wherein the microhollow spheres (16) in the coating (14) are present in a volumetric amount V, with 5% by vol.≦V≦25% by vol.

6. The apparatus according to claim 5, wherein the microhollow spheres (16) in the coating are present in a volumetric amount V, with ≈10% by vol.

7. The apparatus according to claim 1, wherein the microhollow spheres (16) comprise a polymer.

8. The apparatus according to claim 7, wherein the microhollow spheres (16) comprise vinylidine chloride.

9. The apparatus according to claim 1, wherein the microhollow spheres (16) are filled with gas.

10. The apparatus according to claim 9, wherein the microhollow spheres (16) are filled with isobutane.

11. The apparatus according to claim 1, wherein the coating (14) has a total thickness d with 50 μm≦d≦70 μm.

12. The apparatus according to claim 1, wherein the coating (14) comprises at least one layer applied onto the surface (12) of the apparatus (10), which surface is degreased by vapor in a solvent cleaning bath and/or is pretreated with plasma.

13. The apparatus according to claim 12, wherein the coating (14) is applied onto the surface (12) of the apparatus (10) by spraying.

14. The apparatus according to claim 1, wherein the apparatus is a puncture needle, a cannula or a catheter.

15. An echogenic layer (14) comprising a polymer with hollow spaces (16) present therein, wherein the hollow spaces (16) are microhollow spheres (16) embedded in the polymer.

16. The echogenic layer according to claim 15, wherein the polymer is a dispersion of polyurethane or contains polyurethane.

17. The echogenic layer according to claim 15, wherein the microhollow spheres (16) have a diameter D with 5 μm≦D≦50 μm.

18. The echogenic layer according to claim 17, wherein the microhollow spheres (16) have a diameter D with D≈20 μm.

19. The echogenic layer according to claim 15, wherein the microhollow spheres (16) in the layer (14) are present in a volumetric amount V, with 5% by vol.≦V≦25% by vol.

20. The echogenic layer according to claim 19, wherein the microhollow spheres (16) are present in a volumetric amount V, with ≈10% by vol.

21. The echogenic layer according to claim 15, wherein the microhollow spheres (16) comprise a polymer.

22. The echogenic layer according to claim 21, wherein the microhollow spheres (16) comprise vinylidine chloride.

23. The echogenic layer according to claim 15, wherein the microhollow spheres (16) are filled with a gas.

24. The echogenic layer according to claim 23, wherein the microhollow spheres (16) are filled with isobutane.

25. The echogenic layer according to claim 15, wherein the layer (14) has a thickness d, with 50 μpm≦d≦70 μm.
